(19) United States
(12) Patent Application Publication
(10) Pub. No.: US 2024/0319056 A1
(43) Pub. Date: Sep. 26, 2024

(54) Automated Pressuremeter Inflation-Deflation Controller System to Determine in Situ Soil Strength and Deformation Properties

(71) Applicant: Cosentino Engineering Instrumentation Services, LLC, Palm Bay, FL (US)

(72) Inventors: Paul J. Cosentino, Palm Bay, FL (US); Thaddeus J. Misillo, III, Palm Bay, FL (US)

(21) Appl. No.: 18/613,944

(22) Filed: Mar. 22, 2024

Related U.S. Application Data
(60) Provisional application No. 63/454,119, filed on Mar. 23, 2023.

(51) Int. Cl. G01N 3/12; G01N 3/06; G01N 33/24

(52) U.S. Cl. CPC G01N 3/12; G01N 3/064; G01N 33/24; G01N 2203/0048; G01N 2203/0075; G01N 2203/0082; G01N 2203/0244

(57) ABSTRACT

Devices, apparatus, systems and methods for providing a compact, lightweight, handheld pressuremeter controller to perform a soil balloon test with a probe placed into soil, to determine soil strengths and/or stiffnesses/and/or deformations values of the soil. The controller inflates and deflates the balloon through a tubing a quick connect attachment and internal piston-cylinder and tubing components. The controller has a switch, which when activated allows the fully saturated automated device linear or step motor to inject water into the probe while soil resistance pressures and balloon volumes are recorded. Water, or a similar fluid, is injected into the probe until it reaches the prescribed volume. Once that volume is achieved, the motors are controlled to deflate the balloon and the test data is converted from pressure and volume into stress and strain and then to values for soil stiffness and strength.

### CROSS REFERENCE TO RELATED APPLICATIONS

This application claims the benefit of priority to U.S. Provisional Application Ser. No. 63/454,119 filed Mar. 23, 2023, the entire disclosure of which is incorporated herein by specific reference thereto.

### FIELD OF INVENTION

This invention relates to measuring soil strengths and/or stiffnesses/and/or deformations, and in particular to devices, apparatus, systems, and methods for providing a compact pressuremeter control unit to perform a soil balloon test with a probe placed into the soil, to determine soil strengths and/or stiffnesses/and/or deformations of the soil.

### BACKGROUND AND PRIOR ART

Field compaction quality control (QC) of soils requires a specified density and moisture content to be achieved; however, engineers would be more confident in their designs and construction if they used the in-situ stress-strain behavior to determine the soils' stiffness and strength (i.e., modulus and limit pressure). Nuclear density gauge (NDG) testing, a ⅝^{th} inch diameter radioactive probe is inserted into a ¾ inch diameter 6-, 8-, 10- or 12-inch-deep preformed hole, and the radiation emitted is recorded in an instrumented lead-encased 35-pound unit, to produce the in-situ soil moisture and density. With accessories, NDG equipment is carried in a 50-pound transport box around construction sites. Its testing requires skilled and licensed operators, plus significant logistical tracking, and nuclear regulatory paperwork. Replacing NDG testing with equipment that produces strength and stiffness would be a major cost-effective and engineering improvement.

When placing concrete or hot-mix asphalt, samples obtained for quality control (QC) testing ensure these materials meet specified minimum strength requirements. Concrete and asphalt have fixed densities but have a very large range of strengths. Because of soils' nonlinear stress-strain behavior during loading both the strength and stiffness are critical. As with both asphalt and concrete, various strengths and stiffnesses can be associated with a fixed soil density. However, we fail to measure either stiffness or strength during placement.

FIG. 1A is a graph of data from the prior art device shown in FIG. 2 it displays the initial modulus, Eo (psi) versus soil dry density (pcf). FIG. 1B is a graph of data from the prior art device of FIG. 2, it displays the soil Limit Pressure pL (psi) versus soil dry density (pcf). The data are determined using basic elastic theory which is applied to describe material load-deformation behavior.

The data in FIGS. 1A and 1B show that both the modulus (stiffness) and strength (limit pressure) vary significantly at a given density. For example, at a density of 100 pounds per cubic foot (pcf), stiffness varies from below 200 to over 1400 pounds per square inch (psi), while the strength varies from below 10 to nearly 80 psi.

Soil compaction state of practice QC acceptance criteria has remained the same for over 60 years, as field data, reported in terms of moisture and density are checked against standards based on lab tests. Although lab-based soil compaction testing is relatively simple and consistent; no stress-strain, stiffness or ultimate strength data are derived from it.

The QC test used to validate the field compaction is typically NDG testing, an expensive, government-regulated test with regulations that produce serious logistical problems for all geotechnical and pavement consultants.

NDG equipment initially costs approximately $9,000, while annual licensing costs an additional $2,000 to $2,500. Annual calibrations run an additional $500. Proper storage and paperwork can cost thousands more each year. Finally, disposal of the nuclear source can range from $2,000 to $5,000 depending upon the make and model. Logistically, NDG storage requires several layers of security, and the ability to recharge the equipment. A civil engineering consulting firm reported spending $1,100,000 on NDG paperwork for their 70 gauges in one year, or over $15,000 per gauge.

Nuclear density testing has been the accepted quality control soil compaction test for nearly 6 decades. It is fast and produces reliable moisture and density results. Engineers specify minimum field density levels based on very commonly performed laboratory compaction tests known as Proctor compaction tests.

The typical prior art 50-pound nuclear density gauge (NDG) 2, shown in FIG. 2, has a ⅝^{th} inch diameter radioactive probe that is inserted into a preformed hole made by driving a ¾-inch steel pin between 6- and 12-inches into the soil. The equipment requires charging each night in a locked facility and daily calibrations on a heavy standard calibration block.

However, two significant problems have been ignored. First, density is not strength and stiffness, which is what engineers desire to know. Our geotechnical engineering industry has accepted the concept that higher density implies stronger materials, but strength is not measured by this device. Density does not help engineers clearly understand the material properties of compacted materials, as any material with one density can have a large range of strengths, like concrete with a density of 150 pounds per cubic foot and strengths from 2000 to 20,000 pounds per square inch. Density is merely a qualifier that is easy to measure with these radioactive devices. Secondly, as the name implies, a radioactive probe is used, which leads to a significant amount of regulatory paperwork which translates into significant logistical costs.

These gauges have been stolen or involved in various types of accidents. When these occur, the Nuclear Regulatory Commission (NRC) becomes heavily involved.

Various types of soil testing probes have been proposed over the years, in patents and published patent applications but fail to become a practical solution to determine soil strengths and/or stiffnesses/and/or deformations of the soil. See U.S. Pat. No. 4,326,409 to Hughes and U.S. Pat. No. 4,979,197 to Troxler, Sr. et al., which are incorporated by reference in their entirety. See U.S. Published Patent Applications: 2004/0095154 to Lundstrom et al.; and 2011/0194672 to Troxler, which are incorporated by reference in their entirety.

Thus, the need exists for solutions to the problems with the prior art.

### SUMMARY OF THE INVENTION

A primary objective of the present invention is to provide devices, apparatus, systems and methods for providing and controlling soil testing with a compact pressuremeter to perform a soil balloon inflation and deflation test with a probe placed into soil, to determine soil strengths and/or stiffnesses/and/or deformations of the soil.

A secondary objective of the present invention is to provide a safe, reliable, and fast alternative that produces soil stiffnesses, strengths and deformations, instead of densities based on radiation recordings.

A third objective of the present invention is to provide devices, apparatus, systems, and methods for using a compact, lightweight handheld pressure meter an all-in-one unit that works with 6-, 8-, 10- and 12-inch rubber covered cylindrical probes with injected water volumes up to 150 cubic centimeters, to deliver accurate stress-strain test results in minutes for any soils tested.

This new equipment produces data that can be used for applications other than compaction QC, including designs of various foundations for buildings, evaluations of existing foundations for buildings, and evaluations of shallow water soil strengths.

The new equipment also eliminates any NRC concerns and produces savings for the companies.

A preferred embodiment of the compact portable relatively lightweight soil strength/stiffness pressuremeter system for determining soil strengths and/or stiffnesses/and/or deformations of soil, can include: a controller unit housing a computer, electric motor, tubing with connectors to a fluid supply with pump, and rechargeable battery power supply, a ground engaging probe having a balloon to be placed into soil, and a variable-length conduit attached between the pump in the controller unit and the balloon, wherein the pump-to-pump electric motor pumps fluid through the conduit into the balloon, which is inflated and deflated from the fluid supply, and data is provided to the computer which calculates soil stiffness, soil strength, and soil deformations of the soil.

The conduit can include tubing. The tubing can includes lengths between approximately 30 to approximately 5 feet long.

The electric motor can include stepper or linear motors.

The system can further include a quick connect connection between the variable length conduit and the housing.

The pump can include a cylinder and piston. The system can include a data acquisition card.

The system can further include a switch, on the housing, which when activated allows the motor to inject fluid into the probe while soil resistance pressures and balloon volumes are recorded.

The system can include a handheld transport container with a handle, the container having dimensions of approximately 8 inches by approximately 18 inches by approximately 24 inches.

The container can include an overall weight between approximately 25 to approximately 35 pounds.

The system can include at least one USB or similar port on the housing for allowing said data to be downloaded from the computer.

The system can include a port on the housing for recharging the battery.

The fluid supply can include a water supply.

The data can include soil resistance pressures (in Units such as pounds per square inch or kN per meter squared and balloon volumes (in Units such as cubic inches or cubic centimeters) are recorded.

Another embodiment of the compact portable system for determining soil strengths and stiffnesses and deformations of soil without emitting radiation into surrounding soil, can include a controller unit housing a motor, tubing with connectors to a fluid supply with pump, and rechargeable battery power supply. a ground engaging probe having an inflatable member to be placed into soil, and a conduit attached between the pump in the controller unit and the inflatable member, wherein the electric motor pumps fluid through the conduit into the inflatable member, which is inflated and deflated from the fluid supply, and sensed data is used to provide soil stiffness, soil strength, and soil deformations of the soil, without emitting any radiation into the soil.

The sensed data can include soil resistance pressures (in Units such as pounds per square inch or kN per meter squared and balloon volumes (in Units such as cubic inches or cubic centimeters) are recorded.

The inflatable member can include a balloon.

The compact probe can further include a computer to provide the soil stiffness, the soil strength, and the soil deformations of the soil.

Further objects and advantages of this invention will be apparent from the following detailed description of the presently preferred embodiments which are illustrated schematically in the accompanying drawings.

### BRIEF DESCRIPTION OF THE FIGURES

The drawing figures depict one or more implementations in accordance with the present concepts, by way of example only, not by way of limitations. In the figures, like reference numerals refer to the same or similar elements.

FIG. 1A is a graph of data from the prior art device shown in FIG. 2 in initial modulus, Eo (psi) versus Dry density (pcf).

FIG. 1B is a graph of data from the prior art device of FIG. 1, in Limit Pressure pL (psi) versus Dry density (pcf).

FIG. 2 shows a view of a prior art 50-pound nuclear density gauge (NDG), portable gauge.

FIG. 3 is a side view of the novel controller unit with a conduit/tubing connector to the ground engaging probe.

FIG. 4 is a side cross-sectional view of the controller unit of FIG. 3 without component supports.

FIG. 5 is another side cross-sectional view of the controller unit of FIG. 3 including supports.

FIG. 6A is a side view of a cylindrical support used the supports in FIG. 5.

FIG. 6B is a front view of the cylindrical support of FIG. 6A.

FIG. 7A is a side view of an electrical motor support used in FIG. 5.

FIG. 7B is a front view of the electrical motor support of FIG. 7A.

FIG. 8 is a top view of the controller unit of FIG. 3 excluding supports.

FIG. 9 shows a graph of test data from test durations from 10 to 60 seconds using the novel invention.

### DESCRIPTION OF THE PREFERRED EMBODIMENTS

Before explaining the disclosed embodiments of the present invention in detail it is to be understood that the invention is not limited in its applications to the details of the particular arrangements shown since the invention is capable of other embodiments. Also, the terminology used herein is for the purpose of description and not of limitation.

In the Summary above and in the Detailed Description of Preferred Embodiments and in the accompanying drawings, reference is made to particular features (including method steps) of the invention. It is to be understood that the disclosure of the invention in this specification does not include all possible combinations of such particular features. For example, where a particular feature is disclosed in the context of a particular aspect or embodiment of the invention, that feature can also be used, to the extent possible, in combination with and/or in the context of other particular aspects and embodiments of the invention, and in the invention generally.

In this section, some embodiments of the invention will be described more fully with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will convey the scope of the invention to those skilled in the art. Like numbers refer to like elements throughout, and prime notation is used to indicate similar elements in alternative embodiments.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise specifically noted, articles depicted in the drawings are not necessarily drawn to scale.

The basis for the pressure meter theories is the assumption that the pressure meter probe used in the subject invention causes the soil to expand according to plane strain conditions. Plane strain typically occurs when movements in one direction are significantly longer than the other direction.

The pressure meter probe part of the subject invention is thus considered to be an infinitely long cylinder, expanding uniformly in the radial direction. This assumption allows the soil moduli to be determined based on linear elastic theory according to

$$\begin{matrix}Equation 1 & \\ E = 2 ( 1 + v ) \frac{Δ P}{Δ V} {V}_{m} & ( 1 )\end{matrix}$$

where,
E=elastic modulus
ΔP=change in stress
ΔV=change in volume related to ΔP
Vₘ=average total probe volume over ΔP
Vo=initial volume of probe
V=Poisson's ratio which relates horizontal to vertical strain.

The soil limit pressure is determined from the stress versus strain data as the pressure at which the volume of the cavity doubles that the pressure meter probe is placed.

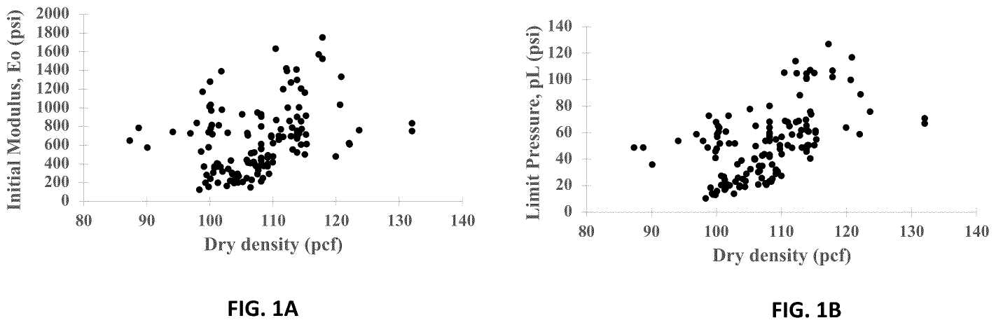

The data in FIGS. 1A and 1B show that both the modulus (stiffness) and strength (limit pressure) vary significantly at a given density. For example, at a density of 100 pounds per cubic foot (pcf), the stiffness varies from below 200 to over 1400 pounds per square inch (psi), while the strength varies from below 10 to nearly 80 psi. See FIGS. 1A and 1B.

Soil compaction state of practice QC acceptance criteria has remained the same for over 60 years, as field data, reported in terms of moisture and density are checked against standards based on lab tests. Although lab-based soil compaction testing is relatively simple and consistent; no stress-strain, stiffness or ultimate strength data are derived from it.

Under Florida Department of Transportation (FDOT) Contract BDV 28 977-04, small diameter pressuremeter (SDPMT) equipment was developed and tested at four locations on and near the Florida Institute of Technology (Florida Tech) campus along Florida's Space Coast. SDPMT probes 6-, 8- 10- and 12-inches in length and ¾-inch in diameter were manufactured. These probes fit in the same hole that is made using the drive pin during NDG testing. During this research, the 6-inch and 12-inch probes were used for testing, enabling both 6- or 12-inch unbound pavement layers to be evaluated.

The invention is a safe, reliable, and fast alternative that produces soil strengths and deformations, instead of densities based on radiation recordings.

A list of the components in the figures will now be described.
PA. prior art 50-pound nuclear density gauge (NDG)
1 Controller unit
2 Connector
3 Tubing
4 Probe
5 power switch
6 USB Charging
8 USB Ports
10 Electric Motor
20 Pump with cylinder
25 piston
30 computer
35 Data Acquisition Card
40 Rechargeable battery
50 Test Status
60 Cylinder Piston Support(s)
70 Electric Motor Support(s)

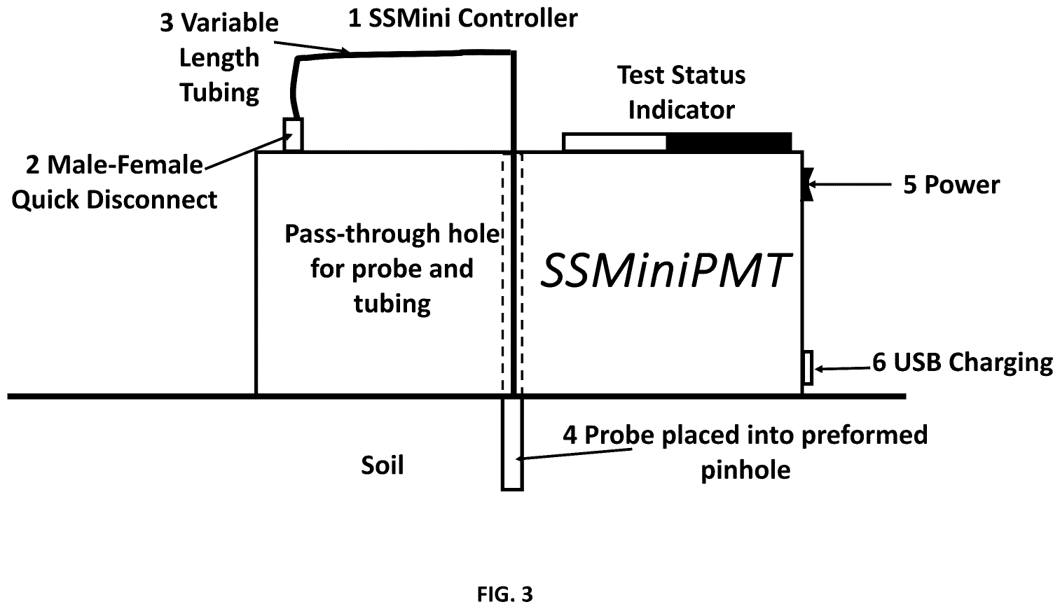

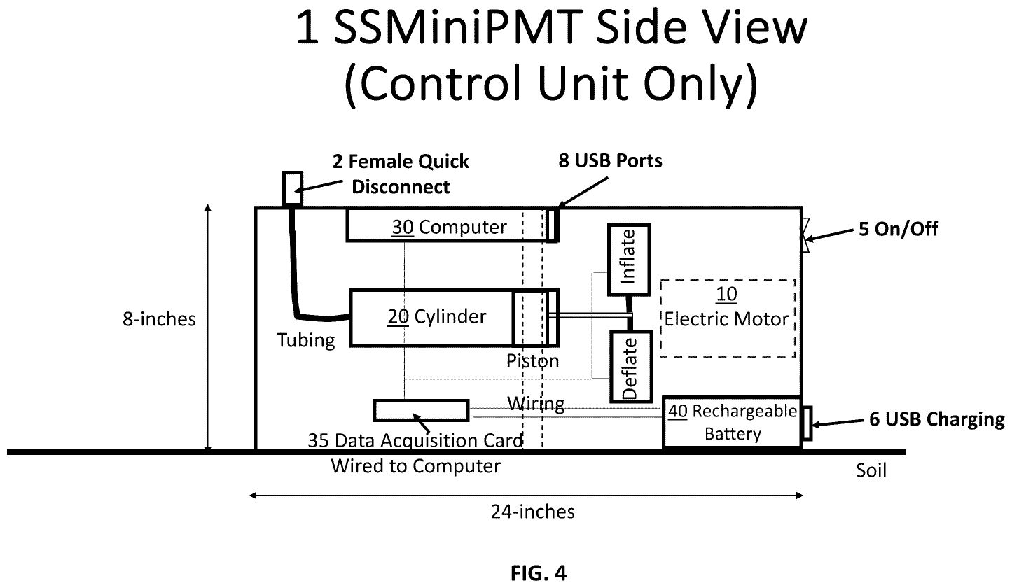

FIG. 3 is a side view of the novel controller unit 1 with conduit/tubing connector 3 to the ground engaging probe 4. FIG. 4 is a side cross-sectional view of the controller unit 1 of FIG. 3 without component supports 60, 70.

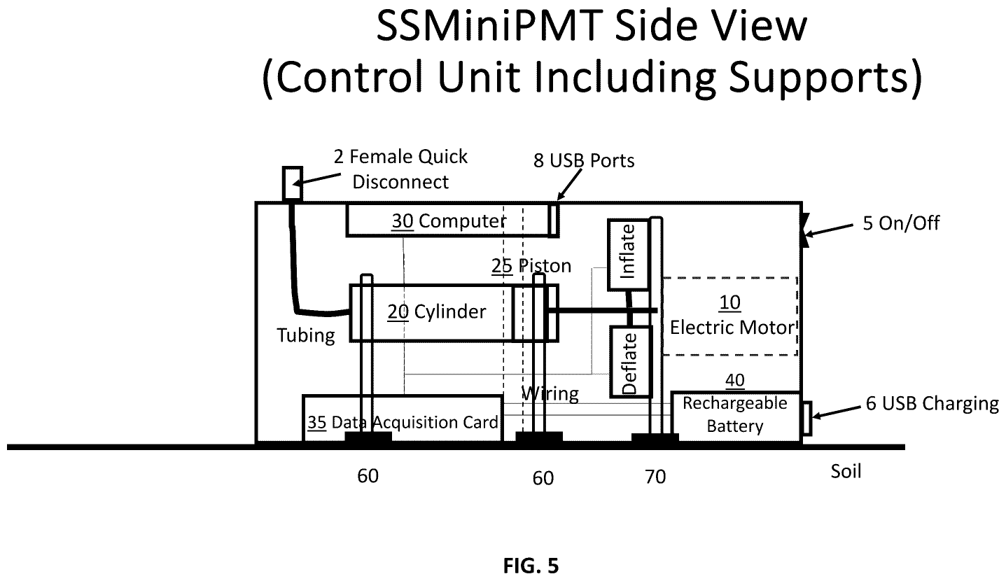

FIG. 5 is another side cross-sectional view of the controller unit 1 of FIG. 3 including Cylinder Piston Supports 60 and Electric Motor Support(s) 70

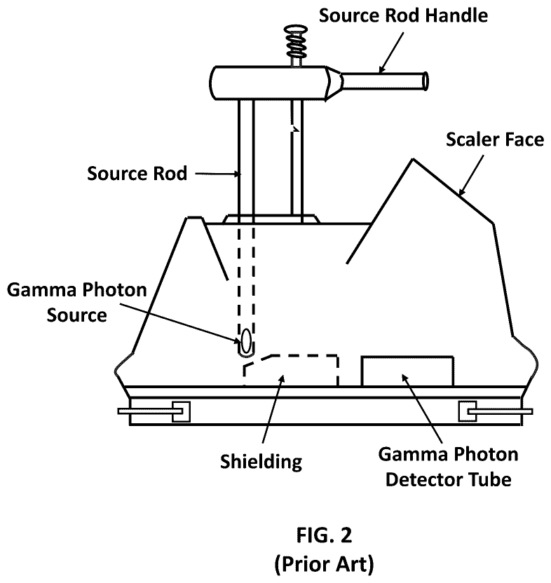

Referring to FIGS. 2-5, the controller unit 1 can include a housing having a height of approximately 8 inches, by approximately 24 inches long by approximately 18 inches wide, and can have an overall weight between approximately 25 lbs. to approximately 35 lbs.

An electric motor 10 such as a stepper or linear motor can operate a pump having a cylinder 20 and piston 25 that pumps a fluid, such as water through a tubing conduit 3 to a ground engaging balloon probe 4, such as but not limited to any current mono-cell pressuremeters, including the PENCEL and TEXAM probes manufactured by Roctest.

A soil balloon test with the controller 1, produces in place soil strengths and/or stiffnesses/and/or deformations within a properly sized hole in the soil, in which the rubber cylindrical balloon 4 is placed, then inflated and deflated.

The controller unit 1 can be equipped with electric step or linear motors 10, a rechargeable battery 40, valves, ¾-in OD tubing 3 such as McMaster Carr Part 51225K42 tubing, a rugged laptop field computer 30 such as a Panasonic Toughbook with software and associated electrical connections and wiring.

The controller 1 has a switch button 5, which when activated allows the fully saturated automated device electric step or linear motor(s) 10 to inject water into the probe 4 while soil resistance pressures (in Units such as pounds per square inch or kN per meter squared and balloon volumes (in Units such as cubic inches or cubic centimeters) are recorded.

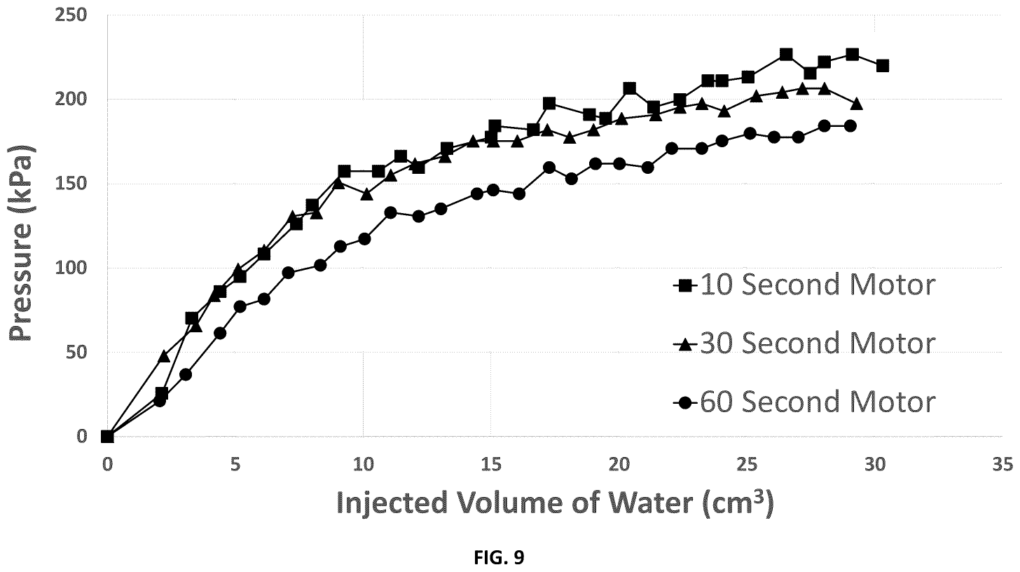

FIG. 9 shows test data from test durations from 10 to 60 seconds. These data are similar to data taken using manual injection methods. The data in FIG. 9 represent tests with the SSMini 6-inch-long probes conducted using a motor to inflate the probe in 10, 30 and 60 seconds.

If these tests are analyzed using equation 1, the data input into the equation along its initial linear response range could be for example Delta P from 50 to 65 kPa or 15 kPa and a Delta V from 2.5 to 4 cubic centimeters or 1.5 cc.

The six-inch long, ⅝-inch diameter probe has an initial volume of about 1.85 cubic inches or 30 cubic centimeters. Using these values and a mean volume of 33.25 cc, with a Poisson's ratio of ½, equation 1 would predict a modulus of 980 kPa. If this process is repeated with Poisson's ratio of ½ for the 30 second motorized test, between 60 and 80 kPa and corresponding volumes of 3 and 4.5 cc, the mean volume of 33.75 cc produces an elastic modulus of 1350 kPa. Likewise for the 60-second motorized test, Poisson's ratio of ½, pressures of 35 and 60 kPa, at volumes of 3 and 5 cc and a mean volume of 34 cc, produce an elastic modulus of 1275 kPa.

A rugged laptop field computer 30, such as but not limited to a Panasonic Toughbook or Dell Latitude 5430 will be used in conjunction with a data acquisition card 35 such as the National Instruments 6000 series USB Multifunction Data Acquisition Device to record all pressures and volumes necessary to properly conduct equipment saturations, calibrations, and testing.

A stainless steel ¾-inch diameter quick-connect such as those available from but not limited to HD Supply can be used to connect and disconnect the tubing from the pump.

A USB rechargeable battery 40 with an indicator light showing when fully charged, can be used such as but not limited to lithium batteries available from DeWalt, Black and Decker, and others can be used to supply power to the electric motor 10 and computer 30. The battery 40 can be approximately 20 volts. Extra ports, such as a side USB port 8 can be used to recharge the battery 40. USB ports 8 are used for data transfer during testing, while USB charging 6 is used to recharge the internal rechargeable batteries 40.

FIG. 5A is a side view of the approximately 4-inch-tall cylinder-piston injection system supports 60 used as the supports 60 in FIG. 4. FIG. 5B is a front view of the approximately 4-inch-tall cylinder support of FIG. 5A. These metallic or plastic components will be attached to the base of the box containing the equipment and secure the cylinder-piston assembly 20,25, enabling both proper transport and testing.

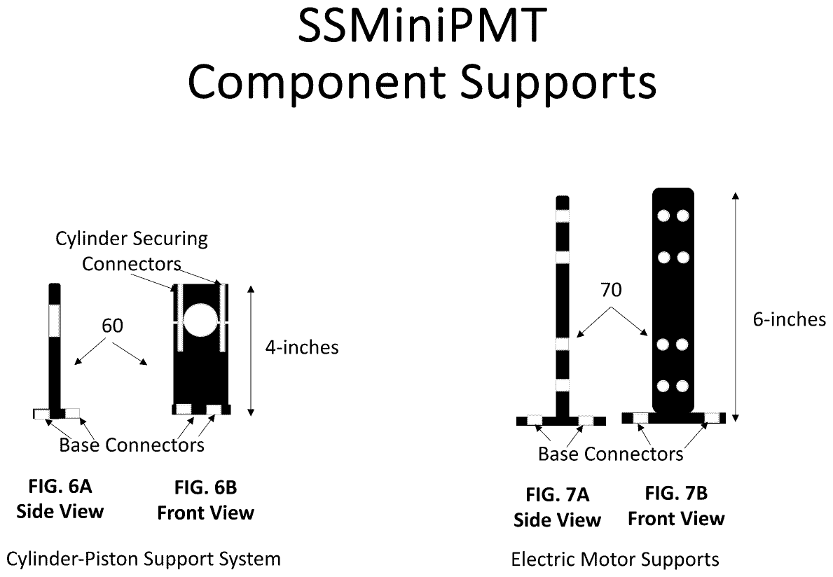

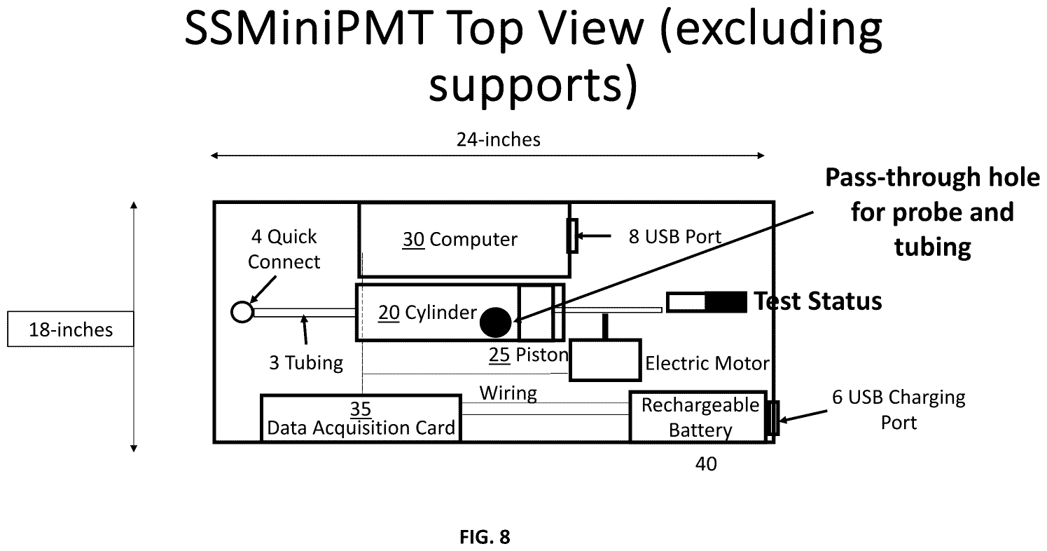

FIG. 6A is a side view of an electric linear or step motor system used to move the piston 25 within the cylinder 20 during probe inflation and deflation motor support 70 used in FIG. 4.

The data acquisition hardware 60 will be programmed to produce these movements at the specified rate allowing tests to be completed within minutes after probe insertion. FIG. 6B is a front view of the electrical motor support 70 of FIG. 6A, it has a group of small openings for bolt attachment to the motor(s) 10.

FIG. 7 is a top view of the controller unit 1 of FIG. 2 excluding supports 60, 70. It details the USB port 6 that is available for instant download of the test data, the test status lights and the relative locations of the internal equipment, i.e., computer 30, cylinder-piston assembly 20, 25, data acquisition hardware card 35 and rechargeable battery 40.

Referring to FIGS. 2-7, a soil balloon test with the controller 1, can produce in place soil strengths and/or stiffnesses/and/or deformations within a properly sized hole in the soil, in which the rubber cylindrical balloon is placed, then inflated and deflated.

Soil strength in soils varies based on the soil composition. Clays are typically the weakest soils and consequently cause the more engineering problems when building on, in or with them. Civil engineers build most structures to resist a maximum settlement of about 1-inch, therefore the soil stiffness which relates settlement to the loads applied by the structure is critical.

The entire controller 1 can be waterproof sealed from the environment, and that can operate in extreme heat up to approximately 110 degrees F. and cold conditions down to approximately 20 degrees F., throughout the workday.

Water, or a similar fluid, can be injected into the probe until it reaches the prescribed volume. Once that volume is achieved, the step motors are controlled to deflate the balloon, and the test data is converted to soil stiffness and strength.

Software can be used to first control the electronic motors, then record the soil pressures on the cylindrical balloon placed in the proper sized test hole, will produce the soils resistance pressures as the probe volume is increased and decreased.

The entire set of data can be stored and can be downloaded to various computer and cloud-based storage devices for immediate use by project managers.

Unlike density and moisture, this set of soil repose data can easily be used to map the variations in soil strength and stiffness along the site. Strength ensures that the soil will not fail under the applied loads while stiffness can be used directly to evaluate possible movements of various structures, such as shallow concrete footings, buried drainage or other utility pipes etc.

Q The software can have subroutines that allow a) indicators that the equipment is calibrated, b) indicators that the internal gauges are functioning properly, c) indicators that the equipment is saturated, d) indicators/screens showing storage of the tubing and membrane calibrations, c) storage of the in-situ test data, and d) control of the motor and cylinder-piston assembly used to inflate and deflate the sealed rubber encased metal probe.

The all-in-one unit can work with 6-, 8-, 10- and 12-inch probes, and will deliver accurate stress-strain test results in minutes for any soils tested. The probes are simply inserted into the same NDG drive-pin hole, the start button is pushed, and the probes are gently inflated and deflated using water to produce stiffness and strength data that is simplified for the field technician into acceptable or unacceptable strengths. This data is also available to the engineers allowing a more detailed analysis.

The controller unit 1 quickly and efficiently produces an in-situ stress-strain curve. The resulting data helps engineers develop a picture of the materials strength-deformation properties across the site. It can be used during classical geotechnical compaction and in the unbound pavement layers of roadways. The ¾-inch diameter probe also allows engineers to evaluate existing pavements by first coring an approximately 1-inch hole then driving the approximately ¾ inch pin into the underlying soils. The 6- to 12-inch probe lengths allow various layer thicknesses to be evaluated.

Stress-strain curves from the controller unit 1 can be tailored to specific engineering needs and may include unloading and reloading loops. Their data can be used to predict elastic moduli at small strains, which can then be compared to either the design resilient moduli or back-calculated resilient moduli.

Because the controller unit 1 testing has the potential to significantly change current practice, the Florida Department of Transportation and several geotechnical consulting companies are assisting with this research.

While the embodiment references a portable computer, the data can be analyzed by a smart phone, and/or by hand to calculate and determine soil stiffness, soil strength, and soil deformations of the soil.

This device can be adapted to test any soil at any location since the tubing connected to the controller can be any length. Tubing could easily be approximately 30 to approximately 50 feet from the unit allowing evaluations in small, hard-to-access locations, such as under buildings, near sinkholes, in caves, and other locations.

Although specific advantages have been enumerated above, various embodiments may include some, none, or all of the enumerated advantages.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

The term “approximately” is similar to the term “about” and can be +/−10% of the amount referenced. Additionally, preferred amounts and ranges can include the amounts and ranges referenced without the prefix of being approximately.

While the invention has been described, disclosed, illustrated, and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

1. A compact portable relatively lightweight soil strength/stiffness pressuremeter system for determining soil strengths and/or stiffnesses/and/or deformations of soil, comprising of:
a controller unit housing a computer, electric motor, tubing with connectors to a fluid supply with pump, and rechargeable battery power supply;
a ground engaging probe having a balloon to be placed into soil; and
a variable-length conduit attached between the pump in the controller unit and the balloon, wherein the pump-to-pump electric motor pumps fluid through the conduit into the balloon, which is inflated and deflated from the fluid supply, and data is provided to the computer which calculates soil stiffness, soil strength, and soil deformations of the soil.

2. The system of claim 1, wherein the conduit includes:
tubing.

3. The system of claim 2, wherein the tubing includes lengths between approximately 30 to approximately 5 feet long.

4. The system of claim 1, wherein the electric motor includes:
stepper or linear motors.

5. The system of claim 1, further comprising:
a quick connect connection between the variable length conduit and the housing.

6. The system of claim 1, wherein the pump includes:
a cylinder and piston.

7. The system of claim 1, further comprising:
a data acquisition card.

8. The system of claim 1, further comprising:
a switch, on the housing, which when activated allows the motor to inject fluid into the probe while soil resistance pressures and balloon volumes are recorded.

9. The system of claim 1, further comprising:
a handheld transport container with a handle, the container having dimensions of approximately 8 inches by approximately 18 inches by approximately 24 inches.

10. The system of claim 8, wherein the container includes an overall weight between approximately 25 to approximately 35 pounds.

11. The system of claim 1, further comprising:
at least one USB or similar port on the housing for allowing said data to be downloaded from the computer.

12. The system of claim 1, further including:
a side port on the housing for recharging the battery.

13. The system of claim 1, wherein the fluid supply includes:
a water supply.

14. The system of claim 1, wherein the data includes:
soil resistance pressures (in Units such as pounds per square inch or kN per meter squared and balloon volumes (in Units such as cubic inches or cubic centimeters) are recorded.

15. A compact portable system for determining soil strengths and stiffnesses and deformations of soil without emitting radiation into surrounding soil comprising of:
a controller unit housing a motor, tubing with connectors to a fluid supply with pump, and rechargeable battery power supply;
a ground engaging probe having an inflatable member to be placed into soil; and
a conduit attached between the pump in the controller unit and the inflatable member, wherein the electric motor pumps fluid through the conduit into the inflatable member, which is inflated and deflated from the fluid supply, and sensed data is used to provide soil stiffness, soil strength, and soil deformations of the soil, without emitting any radiation into the soil.

16. The compact probe of claim 15, wherein the sensed data includes:
soil resistance pressures (in Units such as pounds per square inch or kN per meter squared and balloon volumes (in Units such as cubic inches or cubic centimeters) are recorded.

17. The compact probe of claim 15, wherein the inflatable member includes:
a balloon.

18. The compact probe of claim 15, further comprising:
a computer to provide the soil stiffness, the soil strength, and the soil deformations of the soil.

19. A method for determining soil strengths and stiffnesses and deformations of soil without emitting radiation into surrounding soil comprising the steps of:
providing a ground engaging probe having an inflatable member to be placed into soil;
providing a controller unit housing a motor, tubing with connectors to a fluid supply with pump, and rechargeable battery power supply;
pumping fluid through the conduit into the inflatable member, which is inflated and deflated from the fluid supply; and
sensing data from the probe to provide soil stiffness, soil strength, and soil deformations of the soil, without emitting any radiation into the soil.

20. The method of claim 19, wherein the sensed data includes:
soil resistance pressures (in Units such as pounds per square inch or kN per meter squared and balloon volumes (in Units such as cubic inches or cubic centimeters).
